(19) United States
(12) Patent Application Publication
(10) Pub. No.: US 2020/0200747 A1
(43) Pub. Date: Jun. 25, 2020

(54) APPARATUS TO DETECT TARGET COMPONENTS IN TEST SAMPLES

(71) Applicant: EYE3CONCEPTS INC., Oakville (CA)

(72) Inventors: Rohit CHAND, Oakville (CA); Neha MITTAL, Oakville (CA); Amin RAJABZADEH, Oakville (CA); Seshasai SRINIVASAN, Oakville (CA); David WILSON, Oakville (CA)

(21) Appl. No.: 16/415,925

(22) Filed: May 17, 2019

Related U.S. Application Data
(60) Provisional application No. 62/783,777, filed on Dec. 21, 2018.

(51) Int. Cl. G01N 33/543; G01N 21/64

(52) U.S. Cl. CPC G01N 33/54386; G01N 2021/6439; G01N 2021/6417; G01N 21/6428

(57) ABSTRACT

An example of an apparatus to detect a target component in a test sample is provided. The apparatus includes a sample pad to receive the test sample. The apparatus further includes a conjugate pad in communication with the sample pad to receive the test sample. The test sample is to interact with a first luminescent particle having a protein and a plurality of primary antibodies to form a conjugated sample. A primary antibody binds with the target component. In addition, the apparatus includes an enhancement pad in communication with the conjugate pad to receive the conjugated sample. The conjugated sample is to interact with a second luminescent particle having a chemical molecule which links with the protein to form an enhanced sample. Furthermore, the apparatus includes a membrane in communication with the enhancement pad to receive the enhanced sample. The membrane includes a test region treated with the target component.

### BACKGROUND

Target components in various test samples may be detected using various devices. For example, various chemical components in a sample may be detected using a wide range of chromatography, or various spectrometry methods, such as mass spectrometry or optical spectrometry. Detection of samples using portable devices such as breath analyzers have also been used to detect components such as alcohol above a predetermined threshold in a breath sample.

It is known that the detection of chemical compounds other than ethanol may be desired. For example, testing for the presence of various drugs for enhancing athletic performance may be carried out by various sports regulatory bodies. In other examples, testing for substances that may affect judgement or motor coordination of an individual may be carried out by an employer or regulatory body to improve productivity and reduce the risk of accidents. Similarly, law enforcement agencies may carry out test to detect the presence of banned narcotics.

### BRIEF DESCRIPTION OF THE DRAWINGS

Reference will now be made, by way of example only, to the accompanying drawings in which:

FIG. 1 is a perspective view of an example apparatus to detect a target component in accordance with an example;

FIG. 2 is a perspective view of an example apparatus to detect a target component in accordance with another example;

FIG. 3A is a top view of an example device to detect a target component in accordance with an example;

FIG. 3B is a cross sectional view of the device in FIG. 3A about the line 3-3;

FIG. 4 is graph representing results from the application of a plurality of test samples having different concentrations to an example apparatus;

FIG. 5 is graph representing results from a plurality of example apparatus having different enhancement pad concentrations;

FIG. 6 is graph representing results from a plurality of example apparatus having different amounts of sucrose concentrations to vary the interaction time; and

FIG. 7 is graph representing results to test the effect of amplification across varying THC concentrations.

### DETAILED DESCRIPTION

Lateral flow tests are known and may provide a relatively simple process for testing fluidic samples for various target components. Accordingly, lateral flow tests may be used in numerous applications to carry out chemical and biochemical tests. For example, colored particles or gold nanoparticles may be used for signal generation in some applications; however, lateral flow tests using such particles are typically capable of providing qualitative or semi-quantitative tests. Other particles, such as fluorescent and quantum dots may have a lower cut-off, but these particles are susceptible to photo-bleaching, which may result in low contrast due to auto-fluorescence.

Upconverting nanoparticles are particles that provide a photon upconversion to emit light at a wavelength shorter than the light used to excite the upconverting nanoparticle. For example, an upconverting nanoparticle may emit visible or ultraviolet light when excited with near-infrared light. Upconverting nanoparticles may have long luminescence lifetime, excellent photostability, infrared or near infrared excitation to reduce background noise, and narrow and tunable emission bands to provide a strong signal that may be easily detected. It is to be appreciated by a person of skill in the art with the benefit of this description that a relatively simple device such as a handheld light source with a photodetector may be used to excite and detect the upconverting nanoparticles. In this example, the light source may emit light at a specific wavelength or use a filter to remove wavelengths of light that may otherwise interfere with the detection of a response signal. Similarly, the photodetector is to be configured to detect a specific wavelength of light emitted by the upconverting nanoparticles after excitation, or a filter may be placed in front of the photodetector to remove wavelengths of light that may otherwise interfere with the detection of a response signal. In some examples, the handheld device may be a smartphone with a flash and a camera.

Accordingly, a lateral flow test using upconverting nanoparticles may be used to provide rapid, quantitative, and sensitive detection of target components in fluidic tests samples, such as a fluid from a person. In particular, the lateral flow test may be used to detect tetrahydrocannabinol (THC) in an oral fluid sample.

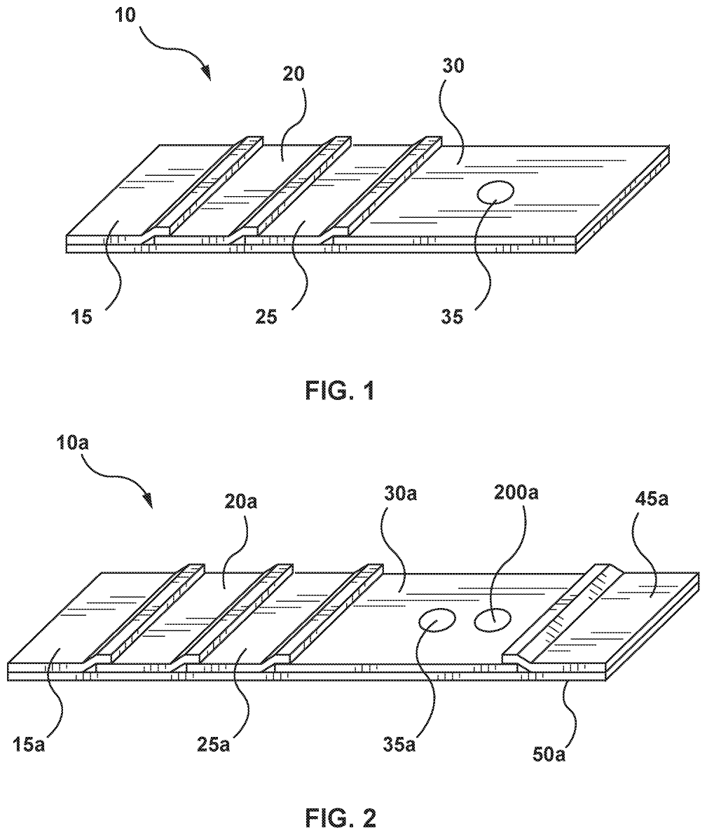

Referring to FIG. 1, an example of an apparatus to detect a target component in a test sample is generally shown at 10. In the present example, the apparatus 10 includes a sample pad 15, a conjugate pad 20, an enhancement pad 25, and a membrane 30. The apparatus 10 may be part of a lateral flow assay device. For example, the apparatus 10 may be enclosed within a housing (not shown) where openings are formed at various locations of the apparatus 10 for receiving a test sample, and/or detection of the test component. For example, the apparatus 10 may be inserted into a reusable housing for each test. In other examples, the apparatus 10 may be encased in the housing such that the lateral flow assay device is to be used once.

The sample pad 15 is to receive a test sample. It is to be appreciated by a person of skill in the art that the sample pad 15 is not particularly limited. In the present example, the sample pad 15 is a cotton fiber pad capable of receiving and absorbing a liquid sample. The sample pad 15 may also be treated with chemicals such as a buffer solution. In other examples the sample pad 15 may be paper, glass fiber, or polyester.

The manner by which the sample pad 15 receives the test sample is also not particularly limited. For example, the test sample may be dropped onto the sample pad using a pipet to measure the volume of the test sample. In other examples, where the exact amount of test sample is not controlled, the test sample may be applied to the sample pad 15 using less precise means, such as pouring the sample onto the sample pad 15, or placing the sample pad into a larger volume liquid such that the sample pad 15 may absorb a test sample.

The conjugate pad 20 is in communication with the sample pad 15 and may receive the test sample via capillary action from the sample pad 15. The material from which the conjugate pad 20 is formed is not limited. In the present example, the conjugate pad 20 is a comprised of glass fibers. However, in other examples, the conjugate pad 20 may be made from cotton fiber, paper, or polyester.

The test sample may interact with components of the conjugate pad 20 to result in a conjugated sample being formed. For example, the conjugated sample may include components that have interacted with the test sample to bind with the test sample prior to continuing along the apparatus via capillary action. It is to be appreciated that the test sample may not bind with any of the components in the conjugate pad 20. For example, the test sample may be chemically inert to the components of the conjugate pad 20 and simply mix with the components in the conjugate pad 20. It is to be appreciated by a person of skill in the art that the conjugated sample refers to the test sample after passing through the conjugate pad 20 whether or not the composition of the sample changes through the interactions with the components of the conjugate pad 20.

The components in the conjugate pad 20 are not particularly limited. In the present example, the conjugate pad 20 includes a plurality of luminescent particles, which may include fluorescent particles. The luminescent particle is not limited and may be any luminescent particle capable of emitting light after absorbing light or other excitant (chemical or electrical) or colored particle. It is to be appreciated that the luminescent particle may absorb light at one wavelength or be excited chemically or electrically and then emit light at another wavelength. Accordingly, the peak emission wavelength of the luminescent particle may be used to detect the presence of the luminescent particle by exciting the luminescent particle and detecting a response within the expected range. In the present example, the luminescent particle may be upconverting nanoparticles.

Furthermore, the luminescent particle may have a probe for a specific target molecule and a protein or chemical linker or aptamer for clustering bonded to the surface of the particle. In this example, probe for specific target molecule may be an antibody such as monoclonal or polyclonal antibody raised in sheep, goats, dogs, horses, chickens, guinea pigs, hamsters, mice, rats, and sheep or chemical probe or aptamers made up of DNA, RNA or proteins may be selected to bind with the target component of the test sample. Accordingly, for a test sample having the target component, the target component may be bound to the luminescent particles via the antibody or chemical probe.

It is to be appreciated by a person of skill in the art with the benefit of this description that if original test sample includes sufficient amount of the target component, each luminescent particle in the conjugate pad 20 will be bound to a target component. Therefore, the amount or concentration of luminescent particles in the conjugate pad may be varied to control a threshold amount of target component to saturate the luminescent particles in the conjugate pad 20. In other examples, the amount of luminescent particles bound to a target component may be measured, such as with a measurement of the luminescence of bound luminescent particles.

The enhancement pad 25 is in communication with the conjugate pad 20 and may receive the conjugated sample via capillary action from the conjugate pad 20. The conjugated sample which includes the test sample and components from the conjugate may interact with components of the enhancement pad 25 to result in an enhanced sample being formed. For example, the enhanced sample may include components that have interacted with the conjugated sample to bind various components. It is to be appreciated that the components that are bound are not particularly limited. For example, the conjugated sample may include luminescent particles with a protein or chemical bonded to the surface of the luminescent particles. The enhancement pad 25 may include additional luminescent particles; however, the luminescent particles of the enhancement pad 25 include a chemical molecule or protein bonded to the surface of the luminescent particles. The molecule attached to the luminescent particles in the enhancement pad 25 is configured to link with the protein or chemical linker or aptamer on the luminescent particles from the conjugate pad 20 to form clusters of chemical linker particles to improve a signal for detection. It is to be appreciate that in other examples, improvements to the signal for detection may be obtained by using other complementary molecules to form the clusters

The enhancement pad 25 enhances the sample detection by linking additional luminescent particles without antibodies to the luminescent particles from the conjugate pad 20 which includes antibodies to bind with the target component. Linking multiple luminescent particles together may enhance the signal provided during luminescence due to the increased number of luminescent particles. In the present example, the luminescent particles in the conjugate pad 20 and the enhancement pad 25 are the same luminescent particles with the exception of having different components on the surface. Accordingly, the luminescent particles from the conjugate pad 20 and the enhancement pad 25 may have substantially the same response to light.

The components in the enhancement pad 25 are not particularly limited. In the present example, the enhancement pad 25 includes a plurality of luminescent particles. The luminescent particle of the enhancement pad 25 is not limited and may be any particle capable of emitting light after absorbing light or any particle capable of altering luminescence of conjugate particle. In the present example, the luminescent particle of the enhancement pad 25 is not limited and may be of the same type as the luminescent particle from the conjugate pad 20.

The membrane 30 is in communication with the enhancement pad 25 and may receive the enhanced sample via capillary action from the enhancement pad 25. In the present example, the membrane 30 includes a test region 35 treated with the target component or a substance similar to the target component or a form of the target component, or an antibody fixed to the test region 35 that would bind to the antibody or the test sample conjugate from the conjugate pad 20. Accordingly, the clusters that pass over the test region 35 will interact with the target component. If the cluster includes antibodies without a bound target component from the test samples, the antibody may bind to the target component in the test region 35. Accordingly, the clusters of the luminescent particles will be concentrated within the test region 35 to provide a good optical signal. Alternatively, if the cluster includes antibodies with a bound target component from the original test samples, the antibodies would not be able to bond with the target component and continue flowing past the test region 35.

It is to be appreciated that variations are contemplated. For example, it is to be appreciated by a person of skill in the art that although FIG. 1 illustrates the sample pad 15, the conjugate pad 20, the enhancement pad 25, and the membrane 30 as separate physical components attached to a substrate, the structure may be substituted with a functionally equivalent structure. For example, the conjugate pad 20 and the enhancement pad 25, and the membrane 30 may be modified to be a single piece of glass fiber having different portions treated separately to form the conjugate pad 20 and the enhancement pad 25 as separate regions on the same physical piece of material.

In other examples, the sample pad 15, the conjugate pad 20, the enhancement pad 25, and the membrane 30 may be modified to be a single unitary piece of glass fiber or membrane material having different portions treated separately to form the sample pad 15, the conjugate pad 20, the enhancement pad 25, and the membrane 30 as separate regions on the same physical piece of material. Further examples may have other combinations on a single piece of material. As yet another example, the sample pad 15, the conjugate pad 20, the enhancement pad 25, and the membrane 30 may be bonded together prior to use to form a single piece of material. Accordingly, by using a single piece, the apparatus 10 be more compact or have an improved form factor. In addition, assembly of the apparatus 10 in the field may also be facilitated since there are fewer parts to put together.

Referring to FIG. 2, another example of an apparatus to detect a target component in a test sample is generally shown at 10a. Like components of the apparatus 10a bear like reference to their counterparts in the apparatus 10, except followed by the suffix “a”. The apparatus 10a includes a sample pad 15a, a conjugate pad 20a, an enhancement pad 25a, a membrane 30a, an absorbent pad 45a and a substrate 50a.

In the present example, the membrane 30a may include a control region 40a. The control region 40a is not particularly limited and may include complementary molecules to capture clusters that move past the test region 35a. Accordingly, the control region 40a may be used to verify the presence of a negative test sample (i.e. a test sample that did not include the target component). In other examples, the signal provided by the clusters of luminescent particles in the control region 40a may be compared with the signal detected in the test region 35a to provide a quantitative measurement of the amount of target component in the original test sample.

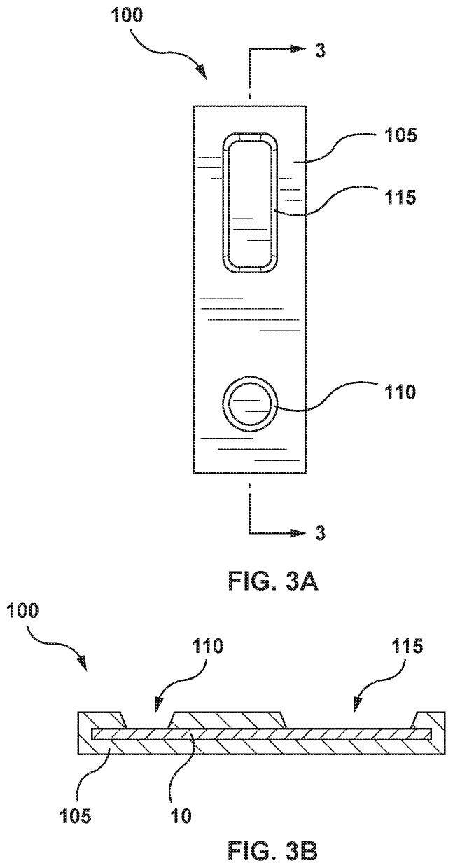

Referring to FIGS. 3A and 3B, a portable device to detect a target component in a test sample is generally shown at 100. In the present example, the portable device 100 is configured to house the apparatus 10. The portable device 100 includes a housing 105 having a sampling window 110 and a detection window 115.

The housing 105 is not particularly limited. For example, the housing 105 may be a unitary body encasing the apparatus 10. In particular, the housing 105 may be a material that is molded around the apparatus 10 such that the apparatus 10 may not be removed from the housing 105. In such an example, the portable device 100 is to be a single use device. In other examples, the housing 105 may include an opening to allow for the insertion and/or removal of the apparatus 10. In such an example, the housing 105 may include two halves configured to mate with each other permanently or separably. In the case that the halves are permanently mated, such that separation would not be easy without breaking the housing 105, the housing 105 may be intended to provide for easy assembly of the portable device 100. In other examples where the housing 105 is separable, the housing 105 may be designed to allow for multiple uses where the apparatus 10 may be exchanged after each use.

It is to be understood that the material of the housing 105 is not particularly limited to any material and that several different types of materials are contemplated. The housing 105 is typically constructed from materials which can easily manufactured such as plastic via an injection molding process. Other types of materials such as metal, glass or composites may also be used.

In the present example, the sampling window 110 is an opening in the housing to allow for the test sample to be dispensed onto the sample pad 15 of the apparatus. The sample pad 15 may also be protected with an optional cover over the sampling window to avoid contamination during storage and/or transportation. The size of the sampling window 110 is also not particularly limited and may vary depending on the specific application of the portable device 100. For example, if the test sample is to be dispensed on the sample pad 15 of the apparatus via an applicator, such as a cotton swab, the sampling window 110 may be dimensioned to approximately the size of the applicator or slightly larger. In particular, some examples of the sampling window may be approximately 0.5 cm to approximately 1.0 cm in diameter. It is to be appreciated by a person of skill in the art that using a smaller sampling window 110 may increase the difficulty of applying the test sample. However, a larger sampling window 110 may increase the amount of contaminants that may be applied to the sample pad 15.

The detection window 115 is to allow for the results of the test sample to be detected. In the present example, the detection window 115 is an opening over the apparatus 10 to expose the test region 35. In other examples, where the apparatus 10a is disposed in the housing, the detection window 115 may be large enough to expose both the test region 35a and the control region 40a. In further examples, the detection window may be divided into separate regions for the test region 35a and the control region 40a.

It is to be appreciated that the detection window 115 may be a physical barrier in some examples, such as a transparent covering. Since the detection of the signals in the test region 35a and the control region 40a is generally done optically, any material that is transparent to the wavelength band where the signal detection is carried out may be used.

### EXAMPLE

An apparatus 10a was prepared to demonstrate the detection of a target component in a test sample qualitatively and quantitatively. In particular, an anti-THC monoclonal antibody conjugated upconverting nanoparticle was used as a selective and sensitive reporter (i.e. luminescent particle) for THC detection. The sensitivity of THC detection of the apparatus 10a was amplified or enhanced by increasing the density of upconverting nanoparticles around THC on the test region 35a. The upconverting nanoparticles were dually conjugated with anti-THC monoclonal antibodies and chemical linker/protein, which in this example is streptavidin. The enhancement pad 25a provides upconverting nanoparticles containing biotin to the test sample. In this example, the enhancement pad 25a was adsorbed with upconverting nanoparticle coated with a biotin as the chemical molecule. It was found that the upconverting nanoparticles tend to cluster on test region 35a to provide an amplified signal with an improved signal-to-noise ratio.

In another example, the upconverting nanoparticles in conjugate pad can be dually conjugated with anti-THC monoclonal antibodies and anti-biotin antibodies. The enhancement pad 25a can be adsorbed with upconverting nanoparticle coated with a biotin as the chemical molecule.

In another example, the upconverting nanoparticles in conjugate pad can be dually conjugated with anti-THC monoclonal antibodies and hemin specific aptamer. The enhancement pad 25a can be adsorbed with upconverting nanoparticle coated with hemin as the chemical molecule.

In the present example, the luminescent particles are upconverting nanoparticles which are conjugated with antibodies, biotin, and/or streptavidin. Depending on the placement of the upconverting nanoparticles within the apparatus 10a, different conjugates may be formed. The manner by which the upconverting nanoparticles are conjugated is not particularly limited and it is to be appreciated by a person of skill in the art with the benefit of this description that alternative conjugates and methods may be used.

For example, upconverting nanoparticles are covalently conjugated to anti-tetreahydrocannabinol antibody and streptavidin (SA) using 1-ethyl-3-(3-dimethylaminopropyl) carbodiimide hydrochloride (EDC) and N-Hydroxysuccinimide (NHS) linkage, commonly known as carbodiimide crosslinking. In particular, the upconverting nanoparticles were dispersed in 4-(2-hydroxyethyl)-1-piperazineethanesulfonic acid (HEPES) buffer with an acidity about pH 6.2 to provide a final concentration of about 0.2 mg/mL. The buffer solution further includes an approximate 2 mM of 1-ethyl-3-(3-dimethylaminopropyl) carbodiimide hydrochloride (EDC) and approximately 5 mM of N-Hydroxysuccinimide (NHS). The mixture was incubated at room temperature with moderate mixing for about 60 min to activate the carboxyl groups of the upconverting nanoparticles. The activated upconverting nanoparticles were then ultra-centrifuged to form pellets. The pellets were reconstituted in HEPES buffer containing about 25 μg of anti-tetreahydrocannabinol antibody and about 5 μg of streptavidin. The reconstituted mixture was incubated for about 4 hours at approximately 23° C. with gentle mixing. The cross-linked upconverting nanoparticles conjugates (UCNP-IgG-SA) were purified using ultra-centrifugation, re-dispersed and stored in the conjugate buffer (about 20 mM HEPES, about pH 7.2, about 1% Tween-20, about 1% Triton X-100, about 1% trehalose, about 5% sucrose, about 0.02% NaN₃ and about 1% bovine serum albumin) and stored at about 4° C. for further use. The upconverting nanoparticle conjugates (UCNP-IgG-SA) were verified using a ultraviolet-visible spectrophotometer.

In another example, the carboxyl activated upconverting nanoparticles can be incubated in 5 μg of anti-tetreahydrocannabinol antibody and about 5 μg of streptavidin. The purified cross-linked upconverting nanoparticles conjugates (UCNP-IgG-SA) can be stored in the conjugate buffer (about 20 mM HEPES, about pH 7.2, about 0.2% Tween-20, about 0.2% Triton X-100, about 1% trehalose, about 5% sucrose, about 0.02% NaN₃ and about 1% bovine serum albumin) and stored at about 4° C. for further use.

Biotin and upconverting nanoparticles were also covalently conjugated in a similar manner as described by mixing the components in an HEPES buffer solution. In particular, the upconverting nanoparticles were initially dispersed in an HEPES buffer along with EDC and NHS. The carboxyl activated upconverting nanoparticles were incubated with about 1 mM of biotin hydrazide solution for about 4 hours at approximately 23° C. with gentle mixing and purified using ultra-centrifugation. The cross-linked upconverting nanoparticles conjugates (UCNPs-B) were stored in conjugate buffer at approximately 4° C. for further use.

In another example, the carboxyl activated upconverting nanoparticles can be incubated in 1 mM of hemin for about 4 hours at approximately 23° C. with gentle mixing and purified using ultra-centrifugation. The cross-linked upconverting nanoparticles conjugates (UCNPs-B) were stored in conjugate buffer at approximately 4° C. for further use.

In the present example, the sample pad 15a is a cotton fiber pad (grade 238) measuring approximately 20 mm long by approximately 4 mm wide. The sample pad 15a is treated with sample buffer of 1X phosphate buffered saline (PBS), about pH 7.4, about 1% bovine serum albumin, about 0.1% Tween-20 and about 0.1% Triton X-100. The sample pad 15a is then dried at about 37° C. for about 12 hours. The conjugate pad 20a is a glass fiber pad measuring approximately 10 mm long by approximately 4 mm wide by approximately 0.5 mm thick. The conjugate pad (GFCP103000) 20a was soaked with the conjugate buffer (about 20 mM HEPES, about pH 7.2, about 1% Tween-20, about 1% Triton X-100, about 1% trehalose, about 5% sucrose, about 0.02% NaN₃ and about 1% bovine serum albumin), followed by drying for about 12 hours at approximately 37° C. Later, the dried conjugate pad 20a was absorbed with UCNPs-IgG-SA and dried at about 37° C. for approximately 1 hour. The enhancement pad 25a is a glass fiber pad (GFCP103000) measuring approximately 10 mm long by approximately 4 mm wide by approximately 0.5 mm thick. The enhancement pad 25a was soaked with the conjugate buffer (about 20 mM HEPES, about pH 7.2, about 1% Tween-20, about 1% Triton X-100, about 1% trehalose, about 5% sucrose, about 0.02% NaN₃ and about 1% bovine serum albumin), followed by drying for about 12 hours at approximately 37° C. Later, the dried enhancement pad 30a was absorbed with UCNPs-B and dried at about 37° C. for approximately 1 hour. The absorbent pad 45a is a cotton fiber pad measuring approximately 20 mm long by approximately 4 mm wide by approximately 2 mm thick. The absorbent pad (grade 320) 45a is to collect fluid after passing through the membrane 30a to avoid the excess accumulation of liquid. Accordingly, the precise dimensions of the absorbent pad 45a may be varied. In addition, variations in the manner by which the various components are prepared are also contemplated. For example, instead of soaking the reagents in the conjugate pad 20a and the enhancement pad 25a, the reagents may be sprayed to better control the amount of reagent in the conjugate pad 20a and the enhancement pad 25a.

The membrane 30a is a nitrocellulose membrane (FF120HP plus) measuring approximately 30 mm long by approximately 4 mm wide by approximately 0.5 mm thick. The test region 35a was manually spotted using a micro-pipette with approximately 0.5 μL of Δ⁹-tetrahydrocannabinol-bovine serum albumin conjugate solution (THC-BSA) having a concentration of about 0.5 mg/mL. The control region 40a was also manually spotted using a micro-pipette with approximately 0.5 μL of goat anti-mouse secondary immunoglobulin antibodies having a concentration of about 2 mg/mL. In the present example, the control region 40a was spotted about 5 mm from the test region 35a. After the spots were applied, the membrane 30a was dried at approximately 37° C. for about 1 hour. Although the test region 35a in the present example is manually spotted, it may be printed in other examples using an automated manufacturing process.

The sample pad 15a, the conjugate pad 20a, the enhancement pad 25a, the membrane 30a, and the absorbent pad 45a were attached on a substrate 50a, such as a backing card. First, the membrane 30a is disposed on the substrate 50a. The absorbent pad 45a and the enhancement pad 25a are disposed on the substrate 50a next such that each of the absorbent pad 45a and the enhancement pad 25a overlap the membrane by about 2 mm to improve the flow of sample between each of the absorbent pad 45a, the enhancement pad 25a, and the membrane 30a. The conjugation pad 20a and the sample pad 15a are then placed on the substrate such that the conjugate pad overlaps the enhancement pad 25a by about 2 mm. Similarly, the sample pad 15a was disposed on the substrate 50a to overlap the conjugate pad 20a by about 2 mm. The apparatus 10a was then to be stored in air-tight containers with a desiccant, such as silica gel to protect from dust and moisture.

Various test samples containing a THC target component in the range of approximately 0 to approximately 50 ng/mL were diluted in running buffer (1X PBS, about 0.1% Tween-20 and about 0.1% Triton X-100). A test sample of approximately 150 μL was pipetted on the sample pad and allowed to laterally flow for approximately 20 minutes. It is to be appreciated that as the test sample moves to the conjugate pad 20, the UCNPs-IgG-SA would form a complex with UCNPs-biotin and with free THC in the test sample or with THC-BSA on the test region 35a. Accordingly, after approximately 20 minutes, the luminescence signal from test region 35a and the control region 40a was measured.

In a test sample containing a threshold amount of THC, the THC interacts with the UCNPs-IgG-SA conjugates to form a UCNPs-IgG-SA-THC complex in the conjugate pad 20a. The UCNPs-IgG-SA-THC complex then reacts with the UCNPs-biotin in the enhancement pad 25a to form a linkage between streptavidin and biotin. Therefore, the two conjugate clusters containing THC would not interact with the THC-BSA fixed in the test region 35a. However, the conjugates clusters are captured on the control region 40a with the pre-fixed goat anti-mouse polyclonal antibody.

Conversely, in a test sample containing no THC or THC below a threshold amount, a majority of the two conjugated clusters free of THC will be captured by the THC-BSA in the test region 35a. Any THC-free conjugates moving past the test region 35a further will moved ahead to be captured by the pre-fixed goat anti-mouse polyclonal antibody in the control region 40a.

For the measurement of luminescence signal, the test region 35a and the control region 40a were excited using a laser source emitting light at a wavelength of approximately 980 nm. The conjugates in the test region 35a and the control region 40a were excited, and the resulting green emission was captured using a camera from a smartphone equipped with an infrared light filter and measured in arbitrary units (“a.u.”). The images were then transferred to a computer and the green intensity was computed using image processing software, which in this case was ImageJ.

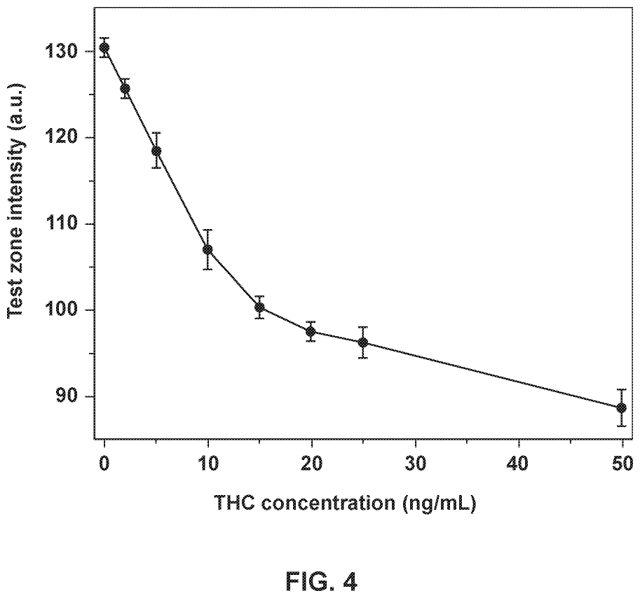

It is to be appreciated that in some applications, the intensity of the luminescent particles, such as the clusters of upconverting nanoparticles, in the test region 35a and the control region 40a may be used to quantify the amount of THC in the original test sample. In such examples, a calibration curve may be generated by plotting the intensity of the luminescent signal from the test region 35a corresponding to a known concentration of THC in a calibration test sample, such as the one shown in FIG. 4. Accordingly, for a test sample of unknown concentration, the quantification of the concentration of THC in the test sample may be estimated using the calibration curve shown in FIG. 4.

In another application of the apparatus 10a, the detection of THC was performed on oral fluids, such as saliva, with added THC. In this example, fresh oral fluid was collected from a healthy person and thoroughly mixed with protease inhibitor cocktail (2 μL/mL). The mixed oral fluid was filtered using a 0.2 μm syringe filter followed by about a 10X dilution with a sample buffer. A known concentration of THC was then added to the diluted oral fluid to spike known concentrations of approximately 10 ng/mL, approximately 25 ng/mL, and approximately 50 ng/mL). The samples were left to incubate for about 30 minutes at room temperature. The samples were then analyzed using the apparatus 10a as discussed above. Table 1 summarizes the detection of THC in the oral fluid. In the present example, each sample was measured three times using a different apparatus 10a for each measurement. The expected signal for each sample was determined based on measurements made with THC in a buffer, such as the results shown in FIG. 4. Difference between the expected signal and the obtained signal may arise due to the interaction with other molecules present in the oral fluid. However, this example provided good correspondence of the spiked THC generally within the estimated margin of error. The results demonstrate the developed UCNPs based LFIA strip may be used for application such as the quantitative detection of THC in oral fluids.

TABLE 1

| Spiked THC | Expected signal | Obtained Signal | % |
|---|---|---|---|
| (ng/mL) | (a.u.) | (a.u.) | Recovery |
| 0 | 130.40 ± 1.12 | 133.45 ± 1.11 | 102.34 |
| 10 | 107.02 ± 2.26 | 107.8267 ± 5.33 | 100.75 |
| 25 | 96.16 ± 1.76 | 96.48 ± 2.05 | 100.34 |
| 50 | 88.70 ± 2.11 | 86.64 ± 2.61 | 97.68 |

### ADDITIONAL EXAMPLES

Several parameters such as sample buffer, membrane type, conjugate concentration, etc. were modified and studied to improve the detection of THC in the test sample. The results were generally analyzed by observing the intensity of light generated by the luminescent particles in the test region 35a.

For example, the preparation of the sample pad 15a and the running buffer used to carry the test sample through the apparatus 10a may be modified to tune the detection characteristics as modifications may change the chemical environment for the immunochemical interaction. Four different buffering systems were compared to obtain better signals. The composition of each buffer and their performances are summarized in Table 2 which provides a qualitative analysis of the apparatus 10a using the different buffering systems. As shown in Table 2, a PBS based buffer system provided the strongest signal in the test region 35a and the control region 40a.

TABLE 2

| Buffer System | Composition | Test Region Intensity |
|---|---|---|
| HEPES | 20 mM HEPES | Weak signal |
| | 0.1% Triton X-100 | |
| | 0.1% Tween 20 | |
| | 1% BSA | |
| | pH 7.4 | |
| Tris-Cl | 20 mM Tris | No signal |
| | 0.1% Triton X-100 | |
| | 0.1% Tween 20 | |
| | 1% BSA | |
| | pH 7.4 using HCl | |
| Tris-E | 20 mM Tris | Weak signal |
| | 1 mM EDTA | |
| | 0.1% Triton X-100 | |
| | 0.1% Tween 20 | |
| | 1% BSA | |
| | pH 7.4 | |
| 1X PBS | 10 mM Phosphate buffer | Strong signal |
| | 137 mM NaCl | |
| | 0.1% Triton X-100 | |
| | 0.1% Tween 20 | |
| | 1% BSA | |
| | pH 7.4 | |

The capillary flow rate and signal intensity of the luminescent particles within the test region 35a were also found to be dependent on the membrane 30a and the size of conjugate used. When a conjugate cluster is formed during the capillary flow, the pore size of the membrane 30a may play a role in delivering the clusters to the test region 35a within the testing period. Variations of the membrane 30a were evaluated using three different membrane types. Each test was performed in triplicates and the qualitative results are summarized in Table 3. The Millipore membrane HF180 produced the least signal intensity perhaps due to the blocking of pores by larger nano-conjugate cluster. The Whatman FF120 plus membrane provided a strong signal due to the larger porosity of membrane.

TABLE 3

| | Test Region | Test Region |
|---|---|---|
| Membranes | Intensity | Signal Shape |
| Millipore HF180 | Weak signal | Incomplete circle |
| Whatman FF170HP | Normal signal | Complete circle |
| Whatman FF120HP Plus | Strong signal | Complete circle |

Variations of the UCNPs-IgG-SA concentration in the conjugate pad 20a was also found to have an effect on the signal intensity at the test region 35a. Although a higher signal intensity allows for easier detection, a higher amount of conjugate in the conjugate pad 20a may provide a false-negative signal while a lower amount of conjugate in the conjugate pad 20a may provide result in a false-positive signal. Therefore, an optimum concentration of conjugate is to be used based on the detection limit of the photodetector used, such as the smartphone camera. To determine a concentration of conjugate to dose the conjugate pad 20a, different conjugate pads were prepared and used to test a test sample having about 50 ng/mL THC. In particular, conjugate pads 20a dosed with different volumes of UCNPs-IgG-SA between approximately 4-15 μL were tested to determine an amount that would provide just a weak signal but not a very bright or diminished signal. The results are summarized in Table 4.

TABLE 4
UCNPs-IgG-SA dosage.

| Dose Volume | Qualitative Test | Qualitative Control |
|---|---|---|
| (in μL) | Region Intensity | Region Intensity |
| 4 | No signal | Weak signal |
| 6 | No signal | Weak signal |
| 8 | Weak signal | Weak signal |
| 10 | Weak signal | Strong signal |
| 15 | Strong signal | Strong signal |

As shown in Table 4, about 4-8 μL of UCNPs-IgG-SA dosage in the conjugate pad 20a produced no signal in the test region 35a which implies the antibodies interacted with the THC in the test sample. By increasing the dosage of UCNPs-IgG-SA in the conjugate pad 20a to about 15 μL of UCNPs-IgG-SA generated a strong signal at the test region 35a, which was comparatively indistinguishable from the signal from the control region 40a. However, a dosage of about 10 μL of UCNPs-IgG-SA generated a faint test signal at the test region 35a and a strong signal at the control region 40a. Accordingly, the clustering of UCNPs at the test region occurs from the interaction between UCNPs-IgG-SA and UCNPs-biotin aided in signal enhancement.

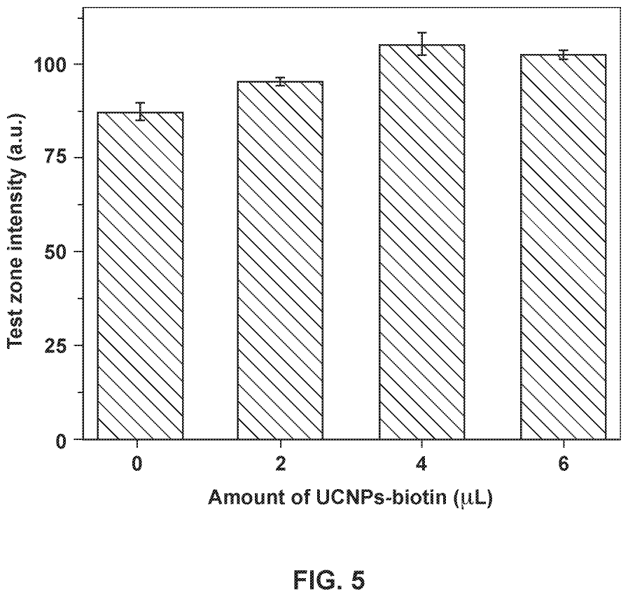

Variations on the dosage of the UCNPs-biotin conjugate in the enhancement pad 25a were also tested. It is to be appreciated by a person of skill with the benefit of this description that a deficiency of UCNPs-biotin results in incomplete cluster formation for signal enhancement. Conversely, an excessive amount of UCNPs-biotin creates a stearic hindrance or bulky cluster to reduce the ability to flow through the membrane 30a. To determine a dosage of UCNPs-biotin to apply to the enhancement pad 25a, different volumes of UCNPs-biotin dosage were prepared. The dosage of UCNPs-IgG-SA in the conjugate pad 20a was fixed at about 10 μL. As shown in FIG. 5, a gradual increase in the signal was seen with an increasing amount of UCNPs-biotin. The maximum signal was obtained with a dosage of approximately 4 μL of UCNPs-biotin in the enhancement pad 25a, beyond which no further increase was obtained.

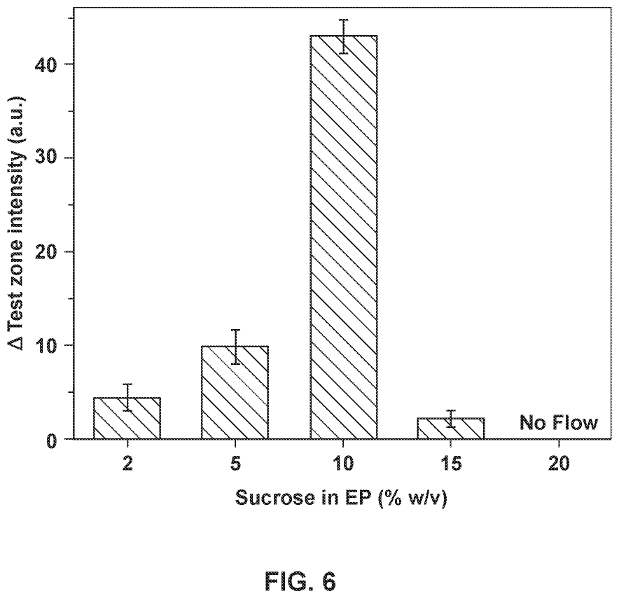

The interaction time of analyte with the conjugates is to be set such that a complete reaction is allowed to occur. In the present example, a sucrose-based obstacle is used to slow the flow of the sample through the apparatus 10a. The dissolution of sucrose results in diffusive mixing of reagents and delays the flow. Accordingly, several concentrations of sucrose in the enhancement pad 25a to extend the residence time of the sample to allow the UCNPs-IgG-SA and UCNPs-biotin conjugates to react with THC in the test sample. Different concentrations (% w/v) of sucrose ranging from 2% to 20% were dissolved in the conjugate buffer and dried on the enhancement pad 25a. The apparatus 10a was then subjected to test samples containing about 0 ng/mL and about 50 ng/mL of THC and the highest reduction in test signal (i.e A intensity) was identified. As shown in FIG. 6, a gradual increase in the signal difference in the test region 35a can be observed, reaching saturation at about 10% sucrose. In apparatus with a ≥20% sucrose in the enhancement pad 25a, it was found that flow of the test sample was completely blocked and no signal was seen in 20 minutes.

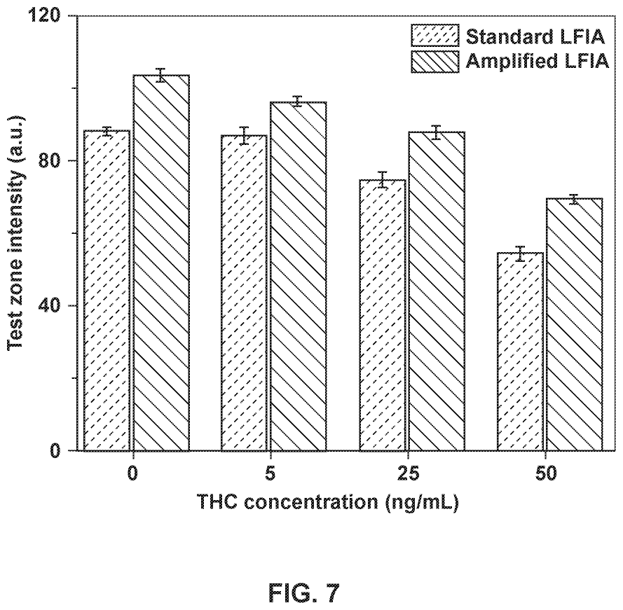

The effects of the UCNPs-biotin in the enhancement pad 25a to enhance the signal of a test sample in the test region 35a was also studied over a range of THC concentrations in the original test sample. Without UCNPs-biotin, conjugate clusters do not form in the test region 35a which causes a decrease in the signal intensity. FIG. 7 shows the comparison of the UCNPs-biotin. As can be seen, an enhanced test signal was obtained through the use of UCNPs-biotin. The clustering of nano-conjugates in the test region 35a as a result of interaction between UCNPs-IgG-SA and UCNPs-biotin aided in signal enhancement by approximately 20%.

It should be recognized that features and aspects of the various examples provided above may be combined into further examples that also fall within the scope of the present disclosure.

What is claimed is:

1. An apparatus to detect a target component in a test sample, the apparatus comprising:
a sample pad to receive the test sample;
a conjugate pad in communication with the sample pad to receive the test sample, wherein the test sample is to interact with a first luminescent particle having a protein and a plurality of primary antibodies to form a conjugated sample, wherein a primary antibody of the plurality of primary antibodies is to bind with the target component;
an enhancement pad in communication with the conjugate pad to receive the conjugated sample, wherein the conjugated sample is to interact with a second luminescent particle having a chemical molecule, wherein the chemical molecule links with the protein to form an enhanced sample; and
a membrane in communication with the enhancement pad to receive the enhanced sample, wherein the membrane has a test region treated with the target component.

2. The apparatus of claim 1, wherein the membrane includes a control region.

3. The apparatus of claim 2, wherein the control region is treated with a secondary antibody, wherein the secondary antibody is to bind with an open primary antibody of the plurality of primary antibodies.

4. The apparatus of claim 1, wherein the first luminescent particle is a first upconverting nanoparticle.

5. The apparatus of claim 4, wherein the second luminescent particle is a second upconverting nanoparticle, wherein the first upconverting nanoparticle and the second upconverting nanoparticle are the same type.

6. The apparatus of claim 1, wherein the protein is streptavidin.

7. The apparatus of claim 6, wherein the first luminescent particle is covalently conjugated to the streptavidin and each of the plurality of primary antibodies.

8. The apparatus of claim 1, wherein the chemical molecule is biotin.

9. The apparatus of claim 8, wherein the second luminescent particle is covalently conjugated to the biotin.

10. A method of detecting a target component in a test sample, the method comprising:
applying the test sample to a sample pad, wherein the test sample is to flow to a conjugate pad, wherein the test sample is to interact with a first luminescent particle having a protein and a plurality of primary antibodies;
binding a primary antibody of the plurality of primary antibodies binds with the target component in the conjugate pad to form a conjugated sample;
linking a chemical molecule having a second luminescent particle with the protein of the first luminescent particle from the conjugated sample in an enhancement pad to form an enhanced sample; and
detecting the target component from the enhanced sample at a test region treated with the target component, wherein detecting the target component involves detecting a presence of the first luminescent particle and the second luminescent particle in the test region.

11. A device to detect a target component in a test sample, the device comprising:
a housing to receive an apparatus to carry out a lateral flow assay test, wherein the apparatus comprises:
a sample pad to receive the test sample;
a conjugate pad in communication with the sample pad, wherein the conjugate pad includes a first luminescent particle having a protein and a plurality of primary antibodies, wherein a primary antibody of the plurality of primary antibodies is to bind with the target component;
an enhancement pad in communication with the conjugate pad, wherein the enhancement pad includes a second luminescent particle having a chemical molecule, the chemical molecule to link with the protein of the first luminescent particle to form a cluster; and
a membrane in communication with the enhancement pad, wherein the membrane has a test region treated with the target component to interact with the cluster based on whether the target component was present in the test sample.
a sampling window above the sample pad to receive the test sample therethrough; and
a detection window to expose the test region to allow measurements taken therethrough.

12. The device of claim 11, wherein the membrane includes a control region.

13. The device of claim 12, wherein the detection window is to expose the control region.

14. The device of claim 12, further comprising a control window to expose the control region to allow measurements taken therethrough.

15. The device of claim 11, wherein the first luminescent particle and the second luminescent particle are the same type.
